(19) United States
(12) Patent Application Publication
(10) Pub. No.: US 2003/0236302 A1
(43) Pub. Date: Dec. 25, 2003

(54) Synthetic derivatives

(76) Inventors: Shoujun Chen, Bedford, MA (US); Lijun Sun, Harvard, MA (US); Joel McCleary, The Plains, VA (US)

Correspondence Address: FISH & RICHARDSON PC, 225 FRANKLIN ST, BOSTON, MA, 02110, US

(21) Appl. No.: 10/376,800

(22) Filed: Feb. 27, 2003

Related U.S. Application Data
(60) Provisional application No. 60/359,864, filed on Feb. 27, 2002.

(51) Int. Cl. A61K 31/366; A61K 31/4433; A61K 31/4025; C 07D 4 5/02; C07D 309/38; C07D 309/30

(52) U.S. Cl. 514/460; 549/292; 549/293; 549/294; 514/336; 514/422; 546/282.4; 548/517

(57) ABSTRACT

This invention features a 5,6-dihydro-2-pyrone compound of formula (I), wherein R₁ is H, OH, or C₂˜C₅ alkoxyl; and R₂ is 2-phenyl ethyl, 2-phenyl ethenyl, 2-heteroaryl ethyl, or 2-heteroaryl ethenyl; in which the phenyl or the heteroaryl is optionally mono-, di-, or tri-substituted with R₃, R₄, or R₅; each of R₃, R₄, and R₅, independently, is Cl, F, Br, I, CN, C₁˜C₅ alkyl, C₁˜C₅ alkoxyl, C₃˜C₅ alkenyloxy, C₄˜C₆ cycloalkoxyl, C₄˜C₈ cycloalkyl alkoxyl, C₃˜C₅ alkoxy alkoxyl, or C₁˜C₄ alkoxy carbonyl.

### CROSS-REFERENCE TO RELATED APPLICATIONS

This application claims benefit of U.S. application serial No. 60/359,864, filed, Feb. 27, 2002, which is herein incorporated by reference in its entirety.

### BACKGROUND

Root extracts from kava (Piper methysticum Forst), a plant found on southern Pacific islands, have been known to induce pleasant and calm feelings, and to treat anxiety or psychological diseases. The psychoactive ingredients of the Kava root have been identified as a class of structurally related chemical compounds known as kavalactones. At least sixteen kavalactones have been identified to date, including kawain, dihydrokawain (a.k.a. marindinin), methysticin, dihydromethysticin, yangonin, and desmethoxyyangonin. These compounds are neutral, nitrogen-poor compounds that may be specifically referred to as substituted alpha.-pyrones. The lactone ring is substituted by a methoxy group in the C-4 position, and the compounds vary in their substitution by either a styryl residue (e.g., yangonin, desmethyoxyyangonin, kawain, and methysticin) or by a phenylethyl residue (e.g., dihydrokawain and dihydromethysticin) at the C-6 position.

Kavalactones have been demonstrated to have anticonvulsive, neuro-protective properties (Kretzschmar et al. (1969) Arch. Int. Pharmacodyn. Ther. 177: 261; and Backhauss et al. (1992) Europ. J. Pharmacol. 215: 265), analgesic effects (Jamieson et al. (1990) Clin. Exp. Pharmacol. Physiol. 17: 495-507), anti-inflammatory effects (Asakawa et al. (2001) Japan KOKAI 2001-316260A), psychoactive effects (Warnecke (1991) Fortschr Med. 109: 119-122), and hair growth inducing/stimulating effects (U.S. Pat. No. 5,585,386). However, the kavalactones are unsuitable for pharmaceutical use due to their short active duration after oral administration, as well as insufficient natural sources. Therefore, it is valuable to the pharmaceutical field to synthesize and characterize novel kavalactone analogs having the aforementioned properties.

### SUMMARY

This invention relates to novel kavalactone analogs (i.e., 5,6-dihydro-2-pyrone compounds), compositions including the analogs, and methods of using the analogs.

The present invention features a 5,6-dihydro-2-pyrone compound of formula (I):

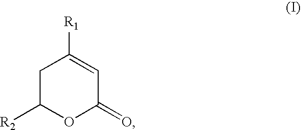

wherein R₁ is H, OH, or C₂˜C₅ alkoxyl; and R₂ is 2-phenyl ethyl, 2-phenyl ethenyl, 2-heteroaryl ethyl, or 2-heteroaryl ethenyl; in which the phenyl or the heteroaryl is optionally mono-, di-, or tri-substituted with R₃, R₄, or R₅; and each of R₃, R₄, and R₅, independently, is Cl, F, Br, I, CN, C₁˜C₅ alkyl, C₁˜C₅ alkoxyl, C₃˜C₅ alkenyloxy, C₄˜C₆ cycloalkoxyl, C₄˜C₈ cycloalkyl alkoxyl, C₃˜C₅ alkoxy alkoxyl, or C₁˜C₄ alkoxy carbonyl.

The heteroaryl is pyryl, indolyl, pyridyl, thienyl, furyl, indazolyl, imidazolyl, thiazolyl, pyrazolyl, or oxazolyl.

In one aspect, the compounds are those of formula (I) wherein R₁ is H. In these compounds, R₂ can be 2-phenyl ethyl, and each of R₃, R₄, and R₅, independently, can be F or OCH₃.

In another aspect, the compounds are those of formula (I) wherein R₁ is OH. In these compounds, R₂ can be 2-phenyl ethyl or 2-heteroaryl ethenyl, and each of R₃, R₄, and R₅, independently, can be F.

In another aspect, the compounds are those of formula (I) wherein R₁ is C₂˜C₅ alkoxyl. In these compounds, R₂ can be 2-phenyl ethyl.

This invention also features a 5,6-dihydro-2-pyrone compound of formula (I), wherein R₁ is OCH₃; and R₂ is 2-phenyl ethyl or 2-phenyl ethenyl; in which the phenyl is mono-, di-, or tri-substituted with R₃, R₄, or R₅; and each of R₃, R₄, and R₅, independently, is F, CN, or C₁˜C₄ alkoxy carbonyl. In some embodiments, each of R₃, R₄, and R₅, independently, is F. In some other embodiments, each of R₃, R₄, and R₅, independently, is CN.

This invention further features a 5,6-dihydro-2-pyrone compound of formula (I), wherein R₁ is OCH₃; and R₂ is 2-heteroaryl ethyl or 2-heteroaryl ethenyl; in which the heteroaryl is optionally mono-, di-, or tri-substituted with R₃, R₄, or R₅; and each of R₃, R₄, and R₅, independently, is Cl, F, Br, I, CN, C₁˜C₅ alkyl, C₁˜C₅ alkoxyl, C₃˜C₅ alkenyloxy, C₄˜C₆ cycloalkoxyl, C₄˜C₈ cycloalkyl alkoxyl, C₃˜C₅ alkoxy alkoxyl, or C₁˜C₄ alkoxy carbonyl. The heteroaryl is pyryl, indolyl, pyridyl, thienyl, furyl, indazolyl, imidazolyl, thiazolyl, pyrazolyl, or oxazolyl.

In another aspect, this invention features a 5,6-dihydro-2-pyrone compound of formula (I), wherein R₁ is H, OH, or C₂˜C₅ alkoxyl; and R₂ is 2-phenyl ethyl, 2-phenyl ethenyl, 2-heteroaryl ethyl, or 2-heteroaryl ethenyl; in which the phenyl or the heteroaryl is optionally mono-, di-, or tri-substituted with R₃, R₄, or R₅; and each of R₃, R₄, and R₅, independently, is Cl, F, Br, I, CN, C₁˜C₅ alkyl, C₁˜C₅ alkoxyl, C₃˜C₅ alkenyloxy, C₄˜C₆ cycloalkoxyl, C₄˜C₈ cycloalkyl alkoxyl, C₃˜C₅ alkoxy alkoxyl, or C₁˜C₄ alkoxy carbonyl; or R₁ is OCH₃; and R₂ is 2-phenyl ethyl or 2-phenyl ethenyl; in which the phenyl is mono-, di-, or tri-substituted with R₃, R₄, or R₅; and each of R₃, R₄, or R₅, independently, is F, CN, or C₁˜C₄ alkoxy carbonyl; or R₁ is OCH₃; and R₂ is 2-heteroaryl ethyl or 2-heteroaryl ethenyl; in which the heteroaryl is optionally mono-, di-, or tri-substituted with R₃, R₄, or R₅; and each of R₃, R₄, and R₅, independently, is Cl, F, Br, I, CN, C₁˜C₅ alkyl, C₁˜C₅ alkoxyl, C₃˜C₅ alkenyloxy, C₄˜C₆ cycloalkoxyl, C₄˜C₈ cycloalkyl alkoxyl, C₃˜C₅ alkoxy alkoxyl, or C₁˜C₄ alkoxy carbonyl. The heteroaryl is pyryl, indolyl, pyridyl, thienyl, furyl, indazolyl, imidazolyl, thiazolyl, pyrazolyl, or oxazolyl.

The term “alkyl” refers to a hydrocarbon chain that may be a straight chain or branched chain, containing the indicated number of carbon atoms. For example, C₁˜C₅ indicates that the group may have from 1 to 5 (inclusive) carbon atoms in it. The term “alkoxyl” refers to an -O-alkyl radical. The term “heteroaryl” refers to an aromatic 5-8 membered monocyclic or 8-12 membered bicyclic ring system comprising one or more heteroatoms (e.g., 0, N, or S). As used herein, the heteroaryl is selected from a group consisting of pyryl, indolyl, pyridyl, thienyl, furyl, indazolyl, imidazolyl, thiazolyl, pyrazolyl, or oxazolyl

The compounds of this invention may contain one or more asymmetric centers and one or more double bonds. Thus, they can occur as racemates and racemic mixtures, single enantiomers, individual diastereomers, diastereomeric mixtures, or cis- or trans or E- or Z-double bond isomeric forms. All such isomeric forms of these compounds are contemplated. All crystal forms of the compounds described herein are expressly included in the present invention.

Exemplary 5,6-dihydro-2-pyrone compounds of this invention are: 6-phenethyl-dihydro-pyran-2,4-dione (Compound 1), 6-[2-(4-fluoro-phenyl)-ethyl]-dihydro-pyran-2,4-dione (Compound 2), 6-[2-(2-fluoro-phenyl)-ethyl]-dihydro-pyran-2,4-dione (Compound 3), 6-[2-(3-fluoro-phenyl)-ethyl]-4 dihydro-pyran-2,4-dione (Compound 4), 6-(2-pyridin-3-yl-vinyl)-dihydro-pyran-2,4-one (Compound 5), 4-methoxy-6-[2-(1H-pyrrol-2-yl)-ethyl]-5,6-dihydro-pyran-2-one (Compound 6), 6-[2-(2-fluoro-phenyl)-ethyl]-4-methoxy-5,6-dihydro-pyan-2-one (Compound 7), 6-[2-(3-fluoro-phenyl)-ethyl]-4-methoxy-5,6-dihydro-pyan-2-one (Compound 8), 6-[2-(4-fluoro-phenyl)-ethyl]-4-methoxy-5,6-dihydro-pyran-2-one (Compound 9), 6-phenethyl-5,6-dihydro-pyran-2-one (Compound 10), 6-[2-(3-fluoro-phenyl)-ethyl]-5,6-dihydro-pyran-2-one (Compound 11), 6-[2-(3,4,5-trimethoxylphenyl)-ethyl]-5,6-dihydro-pyran-2-one (Compound 12), 6-[2-(4-cyano-phenyl)-vinyl]-4-methoxy-5,6-dihydro-pyan-2-one (Compound 13), 6-[2-(3,4-difluoro-phenyl)-vinyl]-4-methoxy-5,6-dihydro-pyan-2-one (Compound 14), 6-[2-(2,3,4-trifluoro-phenyl)-ethyl]-4-methoxy-5,6-dihydro-pyan-2-one (Compound 15), 4-[2-(4-methoxy-6-oxo-3,6-dihydro-2H-pyran-2-yl)-ethyl]-benzoic acid methyl ester (Compound 16), 6-[2-(3,4-difluoro-phenyl)-ethyl]-4-methoxy-5,6-dihydro-pyan-2-one (Compound 17), 6-[2-(4-fluoro-phenyl)-ethyl]-4-methoxy-5,6-dihydro-pyan-2-one (Compound 18), 4-isopropoxy-6-phenethyl-5,6-dihydro-pyran-2-one (Compound 19), 4-ethoxy-6-phenethyl-5,6-dihydro-pyran-2-one (Compound 20), and 4-methoxy-6-(2-pyridin-3-yl-ethyl)-5,6-dihydro-pyran-2-one (Compound 21).

Their structures are shown below.

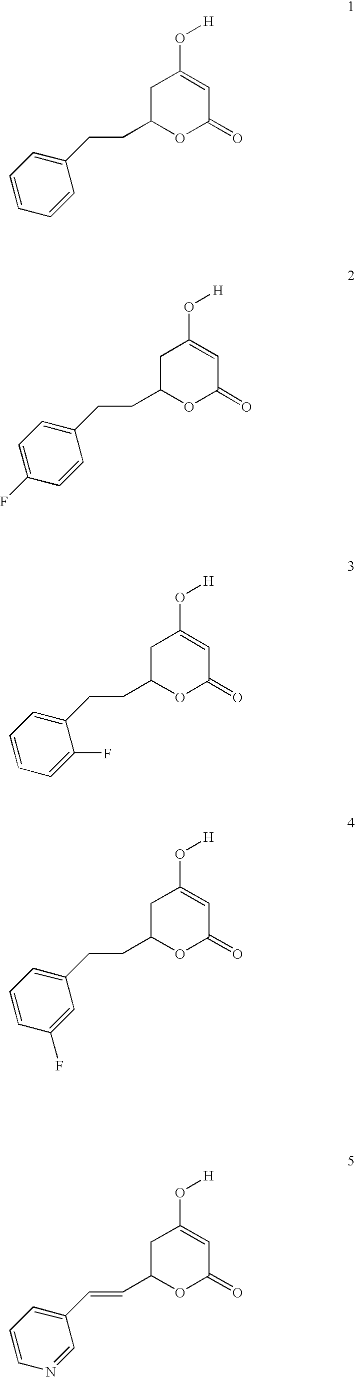

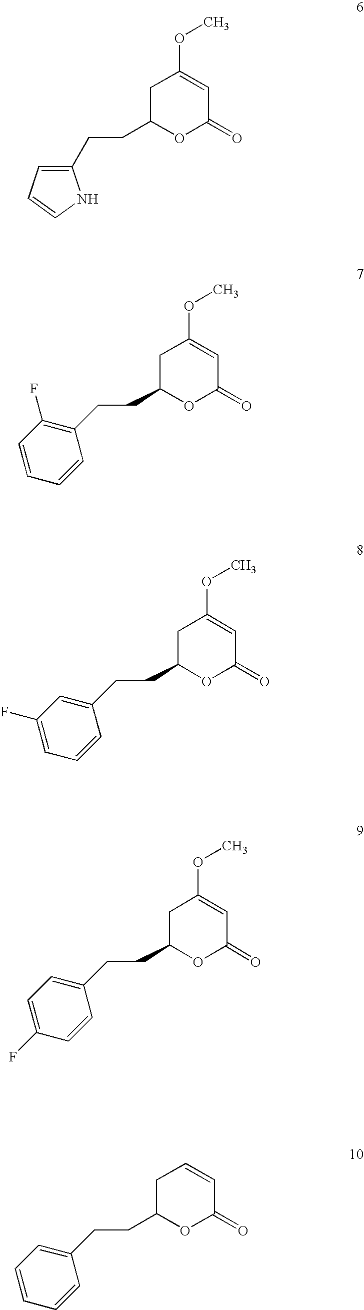

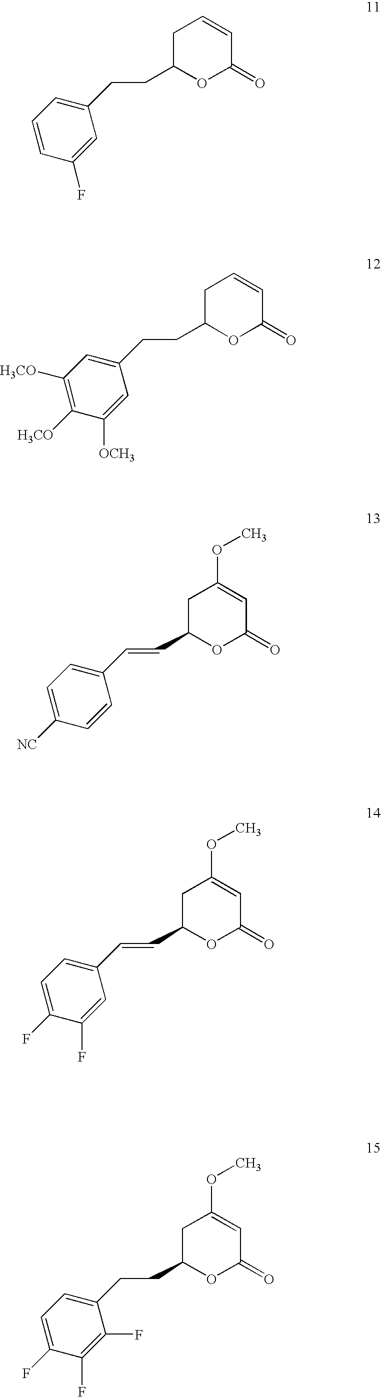

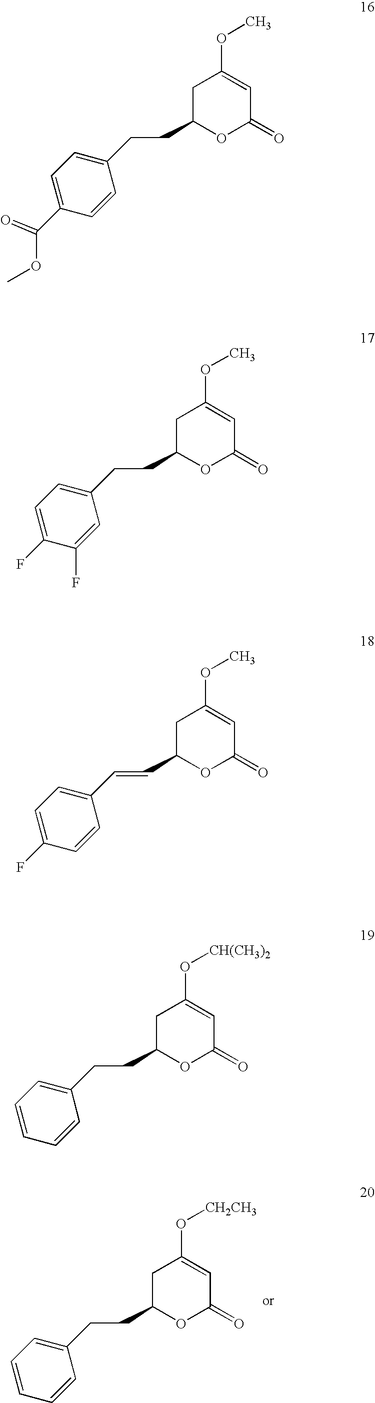

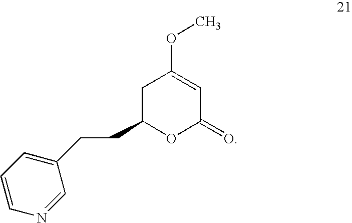

In another aspect, this invention features a pharmaceutical composition comprising one or more compounds described above and a pharmaceutically acceptable carrier.

Other aspects of this invention relate to a composition having a compound of any of the formulae described herein and a pharmaceutically acceptable carrier; or a compound of any of the formulae described herein, an additional therapeutic agent, and a pharmaceutically acceptable carrier.

This invention also features a method for making any of the compounds described above. The method includes reacting a 5-R₂-5-hydroxy-3-oxo-pentanoic acid ester with a reagent to produce any of the compounds of this invention, wherein R₂ is 2-phenyl ethyl, 2-phenyl ethenyl, 2-heteroaryl ethyl, or 2-heteroaryl ethenyl. The method also includes taking any intermediate compound delineated herein, reacting it with any one or more reagents to form a compound of this invention including any processes specifically delineated herein.

This invention further features a method of making a pharmaceutical composition. The method comprises combining one or more compounds of this invention and a pharmaceutically acceptable carrier.

In still another aspect, this invention features a method of treating a disease (e.g., a neurodegenerative disorder, a neurological or psychiatric disorder, pain, or an IL-12 or TNFα-related disorder). The method comprises administering to a subject (e.g., a human, a mammal, or an animal) in need thereof an effective amount of a compound of any of the formulae herein. The IL-12 or TNFα-related disorders include colitis, Crohn's disease, diabetes, encephalomyelitis, multiple sclerosis, oesteoarthritis, periodontitis, psoriasis, rheumatoid arthritis, sepsis, and uveoretinitis.

Further, this invention features a method of eliciting an effect (eliciting an anticonvulsive or antiepileptic effect, eliciting an analgesic effect, inhibiting IL-12 or TNFα production, or stimulating hair growth or retarding hair loss). The method includes administering to a subject in need thereof an effective amount of a compound of any of the formulae herein.

In another aspect, the method includes administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

Also within the scope of this invention is a composition having one or more of the 5,6-dihydro-2-pyrone compounds described above for use in treating the aforementioned diseases, and the use of such a composition for the manufacture of a medicament for the treatment of any of the diseases described herein.

Other features, objects, and advantages of the invention will be apparent from the description and from the claims.

### DETAILED DESCRIPTION

The 5,6-dihydro-2-pyrone compounds (including both optically pure and racemic) described in the “Summary” section can be prepared by methods known in the art. See, e.g., Fowler et al. (1950) J. Chem. Soc. 3642; Isawa et al. (1975) Chem. Lett. 161; Israili et al. (1976) J. Org. Chem. 41: 407; Castellino et al. (1984) Tetrahedron Lett. 25: 4059; Belanger et al (1975) Can. J. Chem. 53: 201; Susuki et al. (1975) Synthesis 192; Achenbach et al. (1970) Tetrahedron Lett. 3259 and 119 (1974); Haensel et al. (1973) Chem. Ber. 106: 570; Nuckin, & Weiler (1974) J. Am. Chem. Soc. 96: 1082; Quallich & Woodall (1993) Synlett 929; Spino, et al. (1996) Tetrahedron Lett. 37: 6503; Israili, & Smissman (1976) J. Org. Chem. 41: 4070; and Izawa, & Mukaiyama (1975) Chem. Lett. 161-164.

The compounds can also be prepared by the synthetic methods disclosed herein. The method includes taking a 5-R₂-5-hydroxy-3-oxo-pentanoic acid ester (R₂ is the same as that is defined in the “Summary” section), and reacting it with one or more chemical reagents in one or more steps to produce a compound of any of the formulae herein. Alternatively, the method includes taking any one of the intermediate compounds described herein and reacting it with one or chemical reagents in one or more steps to produce a compound of any of the formulae described herein. Shown below is a scheme that depicts the just-described synthesis method.

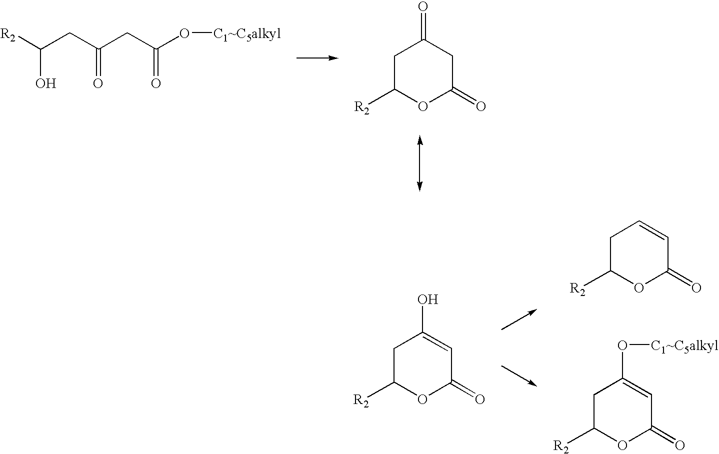

Details of synthesis of the compounds of this invention are described in the Examples. Thus, one embodiment relates to a method of making a compound of the formulae described herein, comprising synthesizing any one or more intermediates illustrated in the synthetic schemes herein and then converting that intermediate(s) to a compound of the formulae described herein. Another embodiment relates to a method of making a compound of the formulae described herein, comprising synthesizing any one or more intermediates illustrated in the examples herein and then converting that intermediate(s) to a compound of the formulae described herein. Another embodiment relates to a method of making a compound of the formulae described herein, comprising synthesizing any one or more intermediates illustrated in the synthetic schemes herein and then converting that intermediate(s) to a compound of the formulae described herein utilizing one or more of the chemical reactions described in the synthetic schemes or examples herein. The chemicals used in the afore-mentioned methods may include, for example, solvents, reagents, catalysts, protecting group and deprotecting group reagents and the like. The methods described above may also additionally comprise steps, either before or after the steps described specifically herein, to add or remove suitable protecting groups in order to ultimately allow synthesis of the compound of the formulae described herein.

As can be appreciated by the skilled artisan, the synthetic schemes herein are not intended to comprise a comprehensive list of all means by which the compounds described and claimed in this application may be synthesized. Further methods will be evident to those of ordinary skill in the art. Additionally, the various synthetic steps described above may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The synthesized compound can be further purified by a method such as column chromatography, high pressure liquid chromatography, or recrystallization.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

As used herein, the compounds of this invention, including the compounds of formulae described herein, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein.

The compounds of this invention may be modified by appending appropriate functionalities to enhance selective biological properties. Such modifications are known in the art and include those which increase biological penetration into a given biological compartment (e.g., blood, lymphatic system, or central nervous system), increase oral availability, increase solubility to allow administration by injection, alter metabolism and alter rate of excretion.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄+salts. This invention also envisions the quaternization of any basic nitrogen-containing groups of the compounds disclosed herein. Water or oil-soluble or dispersible products may be obtained by such quaternization.

Also within the scope of this invention is a pharmaceutical composition that contains an effective amount of at least one compound of any formulae herein and a pharmaceutically acceptable carrier. This invention covers a method of administering an effective amount of one or more compounds of this invention to a subject in need of eliciting an effect (eliciting an anticonvulsive or antiepileptic effect, eliciting an analgesic effect, inhibiting IL-12 or TNFα production, or stimulating hair growth or retarding hair loss). This invention also covers a method of administering an effective amount of one or more compounds of this invention to a subject in need of treating a disease, such as a neurodegenerative disorder, a neurological or psychiatric disorder, pain, or an IL-12 or TNFα-related disorder.

The term “treating” or “treated” refers to administering a compound of this invention to a subject with the purpose to cure, heal, alleviate, relieve, alter, remedy, ameliorate, improve, or affect a disease, the symptoms of the disease or the predisposition toward the disease. “An effective amount” refers to an amount of a compound which confers a therapeutic effect on the treated subject. The therapeutic effect may be objective (i.e., measurable by some test or marker) or subjective (i.e., subject gives an indication of or feels an effect). An effective amount of the compound described above may range from about 1 mg/Kg to about 100 mg/Kg. Effective doses will also vary depending on route of administration, as well as the possibility of co-usage with other agents for treating a disease, including a neurodegenerative disorder, a neurological or psychiatric disorder, pain, or an IL-12 or TNFα-related disorder.

As used herein, the neurodegenerative disorders include Huntington's disease, Parkinson's disease, and Alzheimer's disease.

The neurological or psychiatric disorders include anxiety, a mood disorder (e.g., bipolar disorder, dysthymia, or seasonal effective disorder), and depression.

The term “pain” includes pain itself and a pain-related disorder. It can be complex regional pain syndrome, reflex sympathetic dystrophy, central pain and dysesthesia syndrome, neurogenic pain, Costen's pain-dysfunction, colic, neuropathic pain, or pain asymbolia. Pain conditions include, but are not limited to, pain elicited during various forms of tissue injury, e.g., inflammation, infection, and ischemia; pain associated with musculoskeletal disorders, e.g., joint pain, or arthritis; tooth pain; headaches, e.g., migrane; pain associated with surgery; pain related to inflammation, e.g., irritable bowel syndrome; chest pain; or hyperalgesia, e.g., excessive sensitivity to pain (described in, for example, Fields (1987) Pain, New York:McGraw-Hill).

The IL-12 or TNFα-related disorders include colitis, Crohn's disease, diabetes, encephalomyelitis, multiple sclerosis, oesteoarthritis, periodontitis, psoriasis, rheumatoid arthritis, sepsis, and uveoretinitis.

To practice the method of treating a disease, the compounds, or compositions thereof, can be administered to a patient, for example, in order to treat a disease described above. The compound can, for example, be administered in a pharmaceutically acceptable carrier such as physiological saline, in combination with other drugs, and/or together with appropriate excipients. The compound described herein can, for example, be administered by injection, intravenously, intraarterially, subdermally, intraperitoneally, intramuscularly, or subcutaneously; or orally, buccally, nasally, transmucosally, topically, in an ophthalmic preparation, by inhalation, by intracranial injection or infusion techniques, with a dosage ranging from about 1 to about 100 mg/kg of body weight, preferably dosages between 5 mg and 30 mg/dose, every 4 to 120 hours, or according to the requirements of the particular drug. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

The term “pharmaceutically acceptable carrier” refers to a carrier (adjuvant or vehicle) that may be administered to a patient, together with a compound of this invention, and which does not destroy the pharmacological activity thereof and is nontoxic when administered in doses sufficient to deliver a therapeutic amount of the compound.

Pharmaceutically acceptable carriers that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein. Oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

A sterile injectable composition (e.g., aqueous or oleaginous suspension) can be formulated according to techniques known in the art using suitable dispersing or wetting agents (such as, for example, Tween 80) and suspending agents.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A suitable in vitro assay can be used to preliminarily evaluate a compound of this invention in treating a disease. In vivo screening can also be performed by following procedures well known in the art. See the specific examples below.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

The invention will be further described in the following example. It should be understood that these examples are for illustrative purposes only and are not to be construed as limiting this invention in any manner.

### EXAMPLES

### Example 1

### Synthesis of Compound 1: S-(+)-6-Phenethyl-dihydro-pyran-2,4-dione

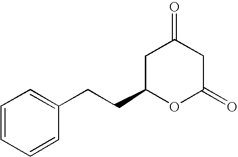

To a stirred solution of (S)-(−)-α,α-diphenyl-2-pyrrolidinemethanol (770 mg, 3 mmol) in dry THF (100 mL) was added 2 M THF solution of borane-dimethylsulfide complex (11 mL, 22 mmol) at room temperature under N₂. After being stirred at room temperature for 16 h, a solution of 3-oxo-5-phenyl-pentanoic acid methyl ester (see, e.g., Nuckin, & Weiler (1974) J. Am. Chem. Soc. 96: 1082) (6.5 g, 31.5 mmol) in dry THF (20 mL) was added dropwise at room temperature for 1 h. The resultant clear solution was stirred at room temperature for another 30 min and was then cooled to 0° C. in an ice bath. The reaction was quenched by adding MeOH (90 mL), and the reaction pot was concentrated under reduced pressure. The residue was taken up with EtOAc (200 mL), and washed successively with H₂O (100 mL), citric acid (100 mL), 5% NaHCO₃ (100 mL), and brine (100 mL). After being dried over Na₂SO₄, the solvent was removed to afford an oil. Flush column chromatography purification on silica gel gave 3-hydroxy-5-phenyl-pentanoic acid methyl ester as an oil (3.3 g, 51%) with 92% e.e. Oudged from the corresponding Mosher's ester). ¹H NMR (300 MHz, CDCl₃) δ 1.6-1.9 (m, 2H), 2.4-2.6 (m, 2H), 2.65-2.9 (m, 2H), 3.05 (d, 1H, J=6), 3.7 (s, 3H), 3.95-4.10 (m, 1H), 7.10-7.35 (m, 5H). ESMS calcd (C₁₂H₁₆O₃): 208.1; found: 209.1 (M+H)⁺.

A solution of 2.0M LDA in heptane/THF/ethylbezene (9.4 mL, 18.8 mmol) was added slowly to a stirred solution of tert-butylacetate (2.18 g, 18.8 mmol) in dry THF (20 mL) under N₂ at −78° C. After being stirred at −78° C. for 25 min, a solution of the above made 3-hydroxy-5-phenyl-pentanoic acid methyl ester (1.3 g, 6.25 mmol) in dry THF (10 mL) was then added through a cannula. The resultant clear solution was stirred at 78° C. for 1 h and then at −55° C. for another 1 h. The reaction was quenched by the addition of 20% aqueous acetic acid (20 mL). Two layers were separated and the aqueous layer was extracted with EtOAc (3×30 mL). Combined organic layer was washed with H₂O (50 mL), brine (50 mL, dried with sodium sulfate, and then concentrated to leave an oil. Flush column chromatography purification on silica gel (4:1 hexane/EtOAc to 2:1 hexane/EtOAc) afforded the intermediate 5-hydroxy-3-oxo-7-phenyl-heptanoic acid tert-butyl ester as a colorless oil (1.74 g, 95%). 1H NMR (300 MHz, CDCl₃) δ 1.45 (s, 9H), 1.6-1.9 (m, 2H), 2.50-2.85 (m, 4H), 3.35 (s, 3H), 3.95-4.10 (m, 1H), 7.10-7.30 (m, 5H). ESMS calcd (C₁₇H₂₄O₄): 292.2; found: 291.2 (M−H)⁺.

A solution of 5-hydroxy-3-oxo-7-phenyl-heptanoic acid tert-butyl ester (0.96 g, 3.3 mmol) and TFA (0.38 g, 3.3 mmol) in DCM (60 mL) was stirred at room temperature for 18 h. Removal of the volatile components under reduced pressure to afford the product 6-phenethyl-dihydro-pyran-2,4-dione as an off white solid (0.56 g, 78%). 1H NMR (300 MHz, CDCl₃) δ 1.9-2.2 (m, 2H), 2.4-2.7 (m, 2H), 2.75-2.95 (m, 2H), 3.5 (q, 2H), 4.55-4.65(m, 1H), 7.10-7.35 (m, 5H). ESMS calcd (C₁₃H₁₄O₃): 218.1; found: 219.1 (M+H)⁺.

### Example 2

### Synthesis of Compound 19: S-(+)-4-isopropoxy-6-phenethyl-5,6-dihydro-pyran-2-one

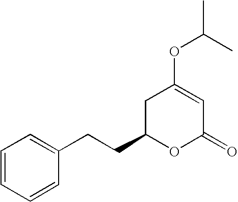

To a stirred solution of 6-phenethyl-dihydro-pyran-2,4-dione (0.22 g, 1.0 mmol) in dry DMF (5 mL) and THF (2 mL) was added NaH (60% in mineral oil, 61 mg, 1.5 mmol). The suspension was stirred at room temperature for 20 min. 2-Iodopropane (0.26 g, 1.5 mmol) was then added and the mixture was stirred at 45° C. for 18 h. The reaction mixture was diluted with EtOAc (80 mL), and washed with H₂O (2×50 mL), brine (50 mL), dried over Na₂SO₄ and concentrated to give an oil. Flush column chromatography purification on silica gel (4:1 hexane/EtOAc to 2:1 hexane/EtOAc) furnished the product S-(+)-4-isopropoxy-6-phenethyl-5,6-dihydro-pyran-2-one as a colorless syrup (0.12 g, 53%). ¹H NMR (300 MHz, CDCl₃) δ 1.3 (m, 6H), 1.85-2.20 (m, 2H), 2.20-2.55 (m, 2H), 2.70-2.95 (m, 2H), 4.3-4.5 (m, 2H), 5.10 (s, 1H), 7.15-7.35 (m, 5H). ESMS calcd (C₁₆H₂₀O₃): 260.1; found: 261.1 (M+H)⁺.

### Example 3

### Synthesis of Compound 2: 6-[2-(4-Fluoro-phenyl)-ethyl]-dihydro-pyran-2,4-dione

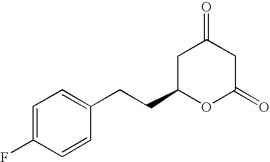

To a stirred suspension of NaH (60% suspension in mineral oil, 1.42 g, 36 mmol) in dry THF (50 mL) was added ethyl acetoacetate (4.3 g, 33 mmol) dropwise at 0° C. After 10 min stirring at this temperature, a 2.0 M solution of n-BuLi in cyclohexane (17.7 mL, 35 mmol) was added dropwise under N₂. The stirring was continued at 0° C. for 10 min, 4-fluorobenzyl bromide (9.45 g, 50 mmol) was then added dropwise at 0° C. The resultant solution was allowed to warm to room temperature. After 20 min, the reaction as quenched with 2 N HCl/Et₂O (20 mL/40 mL). Layers were separated, the aqueous layer was extracted with ether (3×50 mL). Combined ether solution was washed with H₂O (50 mL), brine (50 mL), and dried with Na₂SO₄. After removal of the solvent under reduced pressure, the resultant oily material was purified by flush column chromatography on silica gel (hexane to 4:1 hexane/EtOAc). The corresponding intermediate 5-(⁴-fluoro-phenyl)-3-oxo-pentanoic acid ethyl ester was obtained as a colorless liquid (7.16 g, 92%). ¹H NMR (300 MHz, CDCl₃) δ 1.25 (t, 3H, J=7), 2.80-2.95 (m, 4H), 3.40 (s, 2H), 4.10-4.25 (m, 2H), 6.9-7.0 (m, 2H), 7.1-7.2 (m, 2H). ESMS calcd (C₁₃H₁₅FO₃): 238.1; found: 239.1 (M+H)⁺.

A solution of (S)-(−)-α,α-diphenyl-2-pyrrolidinemethanol (240 mg, 0.97 mmol) and 2 M borane-dimethylsulfide complex (in THF, 3.4 mL, 6.8 mmol) in dry THF (50 mL) was stirred at room temperature under N₂ for 16 h. A solution of 5-(4-fluoro-phenyl)-3-oxo-pentanoic acid ethyl ester (2.03 g, 8.57 mmol) in dry THF (20 mL) was then added dropwise at room temperature over a period of 1 h. The resultant clear solution was stirred at room temperature for another 35 min and was then cooled to 0° C. in an ice bath. The reaction was quenched by the addition of EtOH (40 mL) and was concentrated under reduced pressure. The residue was taken up with EtOAc (100 mL) and washed successively with H₂O (50 mL), 5% NaHCO₃ (50 mL), brine (50 mL), and then dried over Na₂SO₄. Removal of the solvent afforded an oil, which was purified by flush column chromatography on silica gel. The intermediate (S)-5-(4-fluoro-phenyl)-3-hydroxy-pentanoic acid ethyl ester was obtained as a colorless oil (1.33 g, 65%). ¹H NMR (300 MHz, CDCl₃) δ 1.25 (t, 3H, J=7), 1.6-1.9 (m, 2H), 2.35-2.50 (m, 2H), 2.60-2.85 (m, 2H), 3.1 (d, 1H, J=5), 3.95-4.05 (m, 1H), 4.1-4.25 (m, 2H), 6.9-7.0 (m, 2H), 7.05-7.2 (m, 2H). ESMS calcd (C₁₃H₁₇FO₃): 240.1; found: 241.1 (M+H)⁺.

To a stirred solution of tert-butylacetate (1.39 g, 12 mmol) in dry THF (20 mL) was added a solution of 2.0M LDA in heptane/THF/ethylbezene (6 mL, 12 mmol) under N₂ at −75° C. Stirring was continued at −75° C. for 25 min., a solution of (S)-5-(4-fluoro-phenyl)-3-hydroxy-pentanoic acid ethyl ester (0.96 g, 4 mmol) in dry THF (10 mL) was then added through a cannula. The resultant clear solution was stirred at −50° C. for 2 h, quenched by the addition of 20% aqueous acetic acid (20 mL) at 0° C., extracted with EtOAc (3×30 mL). Combined extracts was washed with H₂O (50 mL), brine (50 mL), dried with sodium sulfate, and then concentrated to leave an oil. Flush column chromatography purification on silica gel (hexane to 2:1 hexane/EtOAc) afforded the intermediate 7-(4-fluoro-phenyl)-5-hydroxy-3-oxo-heptanoic acid tert-butyl ester as a colorless oil (0.79 g, 61%). ¹H NMR (300 MHz, CDCl₃) δ 1.45 (s, 9H), 1.6-1.9 (m, 2H), 2.60-2.85 (m, 4H), 3.39 (s, 3H), 3.95-4.15 (m, 1H), 6.95-7.10 (m, 2H), 7.1-7.2 (m, 2H). ESMS calcd (C₁₇H₂₃FO₄): 310.2; found: 309.2 (M−H)⁺.

To a stirred solution of 7-(4-fluoro-phenyl)-5-hydroxy-3-oxo-heptanoic acid tert-butyl ester (0.64 g, 2.06 mmol) in dry DCM was added TFA (0.24 g, 2.1 mmol) at 0° C. The resultant clear solution was stirred at room temperature for 16 h. Removal of the volatile components under reduced pressure afforded the product 6-[2-(4-fluoro-phenyl)-ethyl]-dihydro-pyran-2,4-dione as a white solid (0.32 g, 66%). ¹H NMR (300 MHz, CDCl₃) δ 1.8-2.2 (m, 2H), 2.25-2.55 (m, 2H), 2.65-2.95 (m, 2H), 3.55 (dd, ˜1H, J=10), 4.25-4.40(m, ˜0.5H), 4.6-4.7 (m, ˜0.5H), 5.2 (s, ˜0.5H), 6.90-7.05 (m, 2H), 7.1-7.2 (m, 2H), 10.8 (br, ˜0.5H). ESMS calcd (C₁₃H₁₃FO₃): 236.1; found: 237.1 (M+H)⁺.

### Example 4

### Synthesis of Compound 9: (S)-6-[2-(4-Fluoro-phenyl)-ethyl]-4-methoxy-5,6-dihydro-pyran-2-one

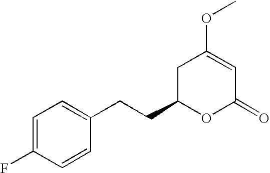

A suspension of K₂CO₃ (0.2 g, 1. 5 mmol), dimethyl sulfate (0.14 g, 1.1 mmol) and 6-[2-(4-fluoro-phenyl)-ethyl]-dihydro-pyran-2,4-dione (0.23 g, 0.97 mmol) in dry acetone was stirred at room temperature for 24 h. Non-dissoluble material was filtered and washed with acetone. Filtrate and washings were combined and concentrated to give an oil. Flush column chromatography purification on silica gel (4:1 hexane/EtOAc to 1:1 hexane/EtOAc) furnished the product (S)-6-[2-(4-fluoro-phenyl)-ethyl]-4-methoxy-5,6-dihydro-pyran-2-one as a white solid (0.15 g, 62%). ¹H NMR (300 MHz, CDCl₃) δ 1.82-2.15 (m, 2H), 2.25-2.60 (m, 2H), 2.70-2.95 (m, 2H), 3.72 (s, 3H), 4.3-4.4 (m, 1H), 5.15 (s, 1H), 6.95-7.05 (m, 2H), 7.12-7.21 (m, 2H). ESMS calcd (C₁₄H₁₅FO₃): 250.1; found: 251.1 (M+H)⁺.

Compounds described in EXAMPLES 5-9 below were synthesized following procedures analogous to the examples described above.

### Example 5

### Synthesis of Compound 3: (S)-6-[2-(2-fluoro-phenyl)-ethyl]-dihydro-pyran-2,4-dione

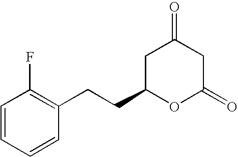

¹H NMR (300 MHz, CDCl₃) δ 1.85-2.2 (m, 2H), 2.4-3.0 (m, 4H), 3.5 (q, 2H), 4.55-4.65(m, 1H), 6.95-7.30 (m, 4H). ESMS calcd (C₁₃H₁₃FO₃): 236.1; found: 237.1 (M+H)⁺.

### Example 6

### Synthesis of Compound 7: (S)-6-[2-(2-Fluoro-phenyl)-ethyl]-4-methoxy-5,6-dihydro-pyran-2-one

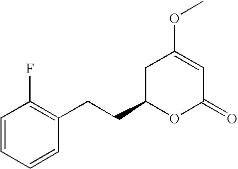

¹H NMR (300 MHz, CDCl₃) δ 1.90-2.18 (m, 2H), 2.25-2.60 (m, 2H), 2.75-3.00 (m, 2H), 3.75 (s, 3H), 4.3-4.4 (m, 1H), 5.15 (s, 1H), 6.95-7.10 (m, 2H), 7.15-7.25 (m, 2H). ESMS calcd (C₁₄H₁₅FO₃): 250.1; found: 251.1 (M+H)⁺.

### Example 7

### Synthesis of Compound 4: (S)-6-[2-(3-Fluoro-phenyl)-ethyl]-dihydro-pyran-2,4-dione

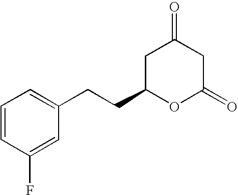

¹H NMR (300 MHz, CDCl₃) δ 1.95-2.2 (m, 2H), 2.45-3.01 (m, 4H), 3.5 (q, 2H), 4.53-4.65(m, 1H), 6.90-7.05 (m, 3H), 7.21-7.35 (m, 1H). ESMS calcd (C₁₃H₁₃FO₃): 236.1; found: 237.1 (M+H)⁺.

### Example 8

### Synthesis of Compound 8: (S)-6-[2-(3-Fluoro-phenyl)-ethyl]-4-methoxy-5,6-dihydro-pyran-2-one

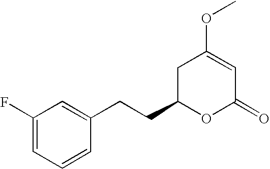

¹H NMR (300 MHz, CDCl₃) δ 1.85-2.18 (m, 2H), 2.25-2.58 (m, 2H), 2.70-2.95 (m, 2H), 3.72 (s, 3H), 4.32-4.41 (m, 1H), 5.18 (s, 1H), 6.85-7.00 (m, 3H), 7.20-7.30 (m, 1H). ESMS calcd (C₁₄H₁₅FO₃): 250.1; found: 251.1 (M+H)⁺.

### Example 9

### Synthesis of Compound 20: (S)-4-Ethoxy-6-phenethyl-5,6-dihydro-pyran-2-one.

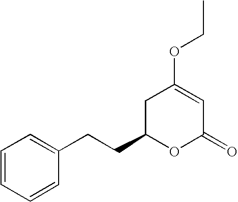

¹H NMR (300 MHz, CDCl₃) δ 1.20 (t, 3H, J=7), 1.85-2.20 (m, 2H), 2.25-2.60 (m, 2H), 2.70-2.95 (m, 2H), 3.84-4.00 (m, 2H), 4.30-4.40 (m, 1H), 5.15 (s, 1H), 7.10-7.35 (m, 5H). ESMS calcd (C₁₄H₁₆O₃): 246.1; found: 247.1 (M+H)⁺.

### Example 10

### Synthesis of Compound 10: (S)-6-Phenethyl-5,6-dihydro-pyran-2-one

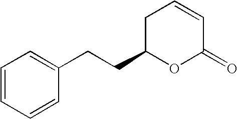

A suspension of (S)-5-hydroxy-3-oxo-7-phenyl-heptanoic acid tert-butyl ester (0.27 g, 0.92 mmol) and NaBH₄ (0.1 g, 2.6 mmol) in THF (15 mL) was stirred under reflux for 12 h. The reaction mixture was allowed to cool to room temperature and poured into ice-H₂O (50 mL), neutralized with concentrated HCl, extracted with EtOAc (3×50 mL), and dried over Na₂SO₄. Removal of the solvent afforded the intermediate 3,5-dihydroxy-7-phenyl-heptanoic acid tert-butyl ester as a white solid (0.27 g, 99%). This material was used directly in the next step.

To a stirred solution of 3,5-dihydroxy-7-phenyl-heptanoic acid tert-butyl ester (0.27 g, 0.92 mmol) in DCM was added TFA (0.11 g, 0.96 mmol) at 0° C. The stirring was continued at room temperature for 16 h. The volatile components were then removed under reduced pressure. The residue was dissolved in benzene (30 mL), TsOH.H₂O (20 mg) was added.

The stirred solution was then heated to reflux for 10 h. During this period, generated H₂O was removed by means of a Dean-Stark trap. The reaction mixture was then allowed to cool to room temperature. Removal of the volatile components afforded an oil which was purified by flush column chromatography on silica gel (4:1 hexane/EtOAc to 1:1 hexane/EtOAc). The corresponding product (S)-6-phenethyl-5,6-dihydro-pyran-2-one was isolated as colorless syrup (0.11 g, 59%). ¹H NMR (300 MHz, CDCl₃) δ 1.90-2.20 (m, 2H), 2.25-2.38 (m, 2H), 2.70-2.95 (m, 2H), 4.35-4.45 (m, 1H), 6.01 (dt, 1H, J=10), 6.80-6.90 (m, 1H), 7.15-7.35 (m, 5H). ESMS calcd (C₁₃H₁₄O₂): 202.1; found: 203.1 (M+H)⁺.

### Example 11

### Synthesis of Compound 11: S-6-[2-(3-Fluoro-phenyl)-ethyl]-5,6-dihydro-pyran-2-one.

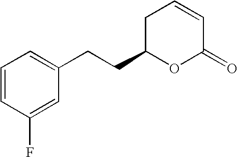

To a stirred suspension of NaH (60% suspension in mineral oil, 1.44 g, 36 mmol) in dry THF (40 mL) was added methyl acetoacetate (3.83 g, 33 mmol) dropwise at 0° C. After 10 min stirring at this temperature, a 2.0 M solution of n-BuLi in cyclohexane (17.5 mL, 35 mmol) was added dropwise under N₂. The stirring was continued at 0° C. for 10 min., 3-fluorobenzyl bromide (9.45 g, 50 mmol) was then added dropwise at 0° C. The resultant solution was allowed to warm to room temperature. After 20 min, the reaction as quenched with 2 N HCl/Et₂O (20 mL/40 mL). Layers were separated, the aqueous layer was extracted with ether (3×50 mL). Combined ether solution was washed with H₂O (50 mL), brine (50 mL), and dried with Na₂SO₄. After removal of the solvent under reduced pressure, the resultant oily material was purified by flush column chromatography on silica gel (hexane to 4:1 hexane/EtOAc). The corresponding intermediate 5-(3-fluoro-phenyl)-3-oxo-pentanoic acid methyl ester was obtained as a colorless liquid (5.68 g, 73%). ¹H NMR (300 MHz, CDCl₃) 62.80-3.00 (m, 4H), 3.41 (s, 2H), 3.72 (s, 3H), 6.85-7.00 (m, 3H), 7.15-7.25 (m, 1H). ESMS calcd (C₁₂H₁₃FO₃): 224.1; found: 225.1 (M+H)⁺.

A solution of (S)-(−)-α,α-diphenyl-2-pyrrolidinemethanol (0.28 g, 1.1 mmol) and 2 M borane-dimethylsulfide complex (in THF, 4.2 mL, 8.2 mmol) in dry THF (40 mL) was stirred at room temperature under N₂ for 15 h. A solution of ⁵-(3-fluoro-phenyl)-3-oxo-pentanoic acid methyl ester (2.68 g, 11.2 mmol) in dry THF (20 mL) was then added dropwise at room temperature over a period of 1 h. The resultant clear solution was stirred at room temperature for another 30 min. The reaction was then quenched by the addition of MeOH (40 mL) at 0° C. and was concentrated under reduced pressure. The residue was taken up with EtOAc (100 mL) and washed successively with H₂O (50 mL), 5% NaHCO₃ (50 mL), brine (50 mL), and then dried over Na₂SO₄. Removal of the solvent afforded an oil, which was purified by flush column chromatography on silica gel (hexane to 4:1 hexane/EtOAc). The intermediate (S)-5-(3-fluoro-phenyl)-3-hydroxy-pentanoic acid methyl ester was obtained as a colorless oil (1.93 g, 75%). 1H NMR (300 MHz, CDCl₃) δ 1.65-1.9 (m, 2H), 2.4-2.6 (m, 2H), 2.65-2.90 (m, 2H), 3.05 (d, 1H, J=6), 3.70 (s, 1H), 3.95-4.05 (m, 1H), 6.85-7.00 (m, 3H), 7.20-7.28 (m, 1H). ESMS calcd (C₁₂H₁₅FO₃): 226.1; found: 227.1 (M+H)⁺.

To a stirred solution of tert-butylacetate (1.68 g, 14.5 mmol) in dry THF (20 mL) was added a solution of 2.0M LDA in heptane/THF/ethylbezene (7.25 mL, 14.5 mmol) under N₂ at −78° C. Stirring was continued at −78° C. for 25 min, a solution of S-5-(3-fluoro-phenyl)-3-hydroxy-pentanoic acid ethyl ester (1.16 g, 4.83 mmol) in dry THF (10 mL) was then added through a cannula. The resultant clear solution was stirred at −55° C. for 3 h, quenched by 20% aqueous acetic acid (20 mL) at 0° C., extracted with EtOAc (3×30 mL). Combined extracts was washed with H₂O (50 mL), brine (50 mL, dried with sodium sulfate, and then concentrated to leave an oil. Flush column chromatography purification on silica gel (hexane to 2:1 hexane/EtOAc) afforded the intermediate 7-(3-fluoro-phenyl)-5-hydroxy-3-oxo-heptanoic acid tert-butyl ester as a colorless oil (1.32 g, 88%). 1H NMR (300 MHz, CDCl₃) δ 1.45 (s, 9H), 1.65-1.85 (m, 2H), 2.60-2.85 (m, 4H), 3.38 (s, 3H), 4.00-4.18 (m, 1H), 6.85-7.00 (m, 3H), 7.20-7.25 (m, 1H). ESMS calcd (C₁₇H₂₃FO₄): 310.2; found: 311.2 (M+H)⁺.

A suspension of 7-(3-fluoro-phenyl)-5-hydroxy-3-oxo-heptanoic acid tert-butyl ester (0.28 g, 0.90 mmol) and NaBH₄ (0.11 g, 2.86 mmol) in THF (15 mL) was stirred under reflux for 5 h. The reaction mixture was allowed to cool to room temperature and poured into ice-H₂O (50 mL), neutralized with concentrated HCl, extracted with EtOAc (3×50 mL), and dried over Na₂SO₄. Removal of the solvent afforded the intermediate 3-ethyl-7-(3-fluoro-phenyl)-3,5-dihydroxy-heptanoic acid tert-butyl ester as an oil (0.28 g, 99%). This material was used directly in the next step. ¹H NMR (300 MHz, CDCl₃) δ 1.45 (s, 9H), 1.65-2.00 (m, 6H), 2.65-2.90 (m, 2H), 3.95-4.15 (m, 1H), 4.30-4.41 (m, 1H), 6.85-7.00 (m, 3H), 7.15-7.25 (m, 1H).

To a stirred solution of 3-ethyl-7-(3-fluoro-phenyl)-3,5-dihydroxy-heptanoic acid tert-butyl ester (0.28 g, 0.90 mmol) in DCM was added TFA (0.11 g, 0.96 mmol) at 0° C. The stirring was continued at room temperature for 18 h. The volatile components were then removed under reduced pressure. The residue was dissolved in benzene (40 mL), TsOH.H₂O (20 mg) was added. A Dean-Stark trap was then equipped and the reaction mixture was then heated to reflux for 18 h. After being cooled to room temperature, the volatile components were removed under reduced pressure to afford an oil, which was purified by flush column chromatography on silica gel (hexane to 2:1 hexane/EtOAc). The S-6-[2-(3-fluoro-phenyl)-ethyl]-5,6-dihydro-pyran-2-one was obtained as colorless syrup (0.08 g, 40%). ¹H NMR (300 MHz, CDCl₃) δ 1.90-2.20 (m, 2H), 2.25-2.45 (m, 2H), 2.70-2.95 (m, 2H), 4.35-4.45 (m, 1H), 6.01 (dt, 1H, J=10), 6.80-7.00 (m, 3H), 7.20-7.31 (m, 1H). ESMS calcd (C₁₃H₁₃FO₂): 220.0; found: 221.0 (M+H)⁺.

EXAMPLES 12-17 illustrate compounds synthesized following procedures analogous to those described above.

### Example 12

### Synthesis of Compound 12: S-6-[2-(3,4,5-trimethoxylphenyl)-ethyl]-5,6-dihydro-pyran-2-one.

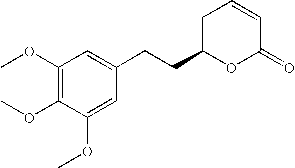

¹H NMR (300 MHz, CDCl₃) δ 1.90-2.20 (m, 2H), 2.25-2.40 (m, 2H), 2.65-2.90 (m, 2H), 3.80 (m, 9H), 4.35-4.45 (m, 1H), 6.01 (dt, 1H, J=10), 6.41 (s, 2H), 6.85-6.96 (m, 1H). ESMS calcd (C₁₆H₂₀O₅): 292.1; found: 293.1 (M+H)⁺.

### Example 13

### Synthesis of Compound 21: 4-Methoxy-6-(2-pyridin-3-yl-ethyl)-5,6-dihydro-pyran-2-one.

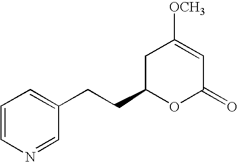

¹H NMR (300 MHz, CDCl₃), 6 (ppm): 8.49 (s, 1H), 8.48 (d, 1H, J=5.4), 7.54 (d, 1H, J=7.8), 7.23 (dd, 1H, J=7.8, 4.8), 5.16 (s, 1H), 4.37 (m, 1H), 3.75 (s, 3H), 2.93 (m, 1H), 2.80 (m, 1H), 2.54 (dd, 1H, J=17.1, 12.0), 2.32 (dd, 1H, J=17.0, 3.8), 2.13 (m, 1H), 1.94 (m, 1H). ESMS calcd (C₁₃H₁₅NO₃): 233.1; found: 234.1 (M+H).

### Example 14

### Synthesis of Compound 5: 6-(2-pyridin-3-yl-vinyl)-dihydro-pyran-2,4-one Mixture of Cis- and Trans-Isomers and Keto- and Enol-Forms

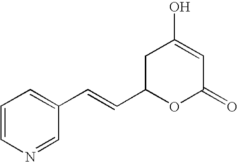

¹H NMR (300 MHz, CDCl₃), 6 (ppm): 8.65 (m, 1H), 8.52 (m, 1H), 7.78 (m, 1H), 7.32 (m, 1H), 6.87 (m, 1H), 6.34 (m, 1H), 5.23 (m, 1H), 3.60 (m, 1H), 2.74 (m, 2H). ESMS calcd (C₁₂H₁₁NO₃): 217.1; found: MS (ESI): 218.1 (M+H).

### Example 15

### Synthesis of Compound 6: 4-Methoxy-6-[2-(1H-pyrrol-2-yl)-ethyl]-5,6-dihydro-pyran-2-one

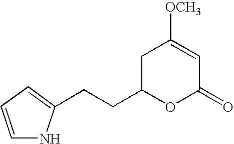

¹H NMR (300 MHz, CDCl₃), 6 (ppm): 8.20 (brs, 1H), 6.68(m, 1H), 6.13 (dd, 1H, J=5.4, 2.7), 5.93(m, 1H), 5.13(s, 1H), 4.60 (m, 1H), 3.73 (s, 3H), 2.82 (m, 2H), 2.50 (dd, 1H, J=17.1, 2.7), 2.30 (dd, 1H, J=17.1, 4.2), 2.09 (m, 1H), 1.94 (m, 1H). ESMS calcd (C₁₂H₁₅NO₃): 219.1; found: 220.1 (M+H)⁺.

### Example 16

### Synthesis of Compound 15

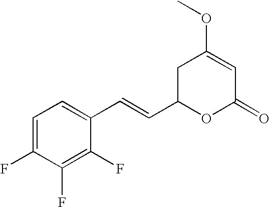

¹H-NMR (CDCl₃) δ 2.66-2.78 (m, 2H), 3.88 (s, 3H), 5.26-5.33 (m, 1H), 5.65 (s, 1H), 6.37 (dd, J₁=5.7 Hz, J₂=15.9 Hz, 1H), 6.89 (d, J₂=15.9 Hz, 1H), 6.96 (d, 6.9 Hz, 1H), 7.24 (d, 6.9 Hz, 1H), ESMS Calcd (C₁₄H₁₁F₃O₃): 284.07, found (M+H)⁺.

### Example 17

### Synthesis of Compound 14

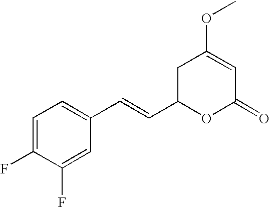

¹H-NMR (CDCl₃) δ 2.54-2.73 (m, 2H), 3.80 (s, 3H), 5.06-5.14 (m, 1H), 5.33 (s, 1H), 6.17 (dd, J₁=5.7 Hz, J₂=15.9 Hz, 1H), 6.72 (d, J₂=15.9 Hz, 1H), 7.08-7.25 (m, 4H), ESMS Calcd (C₁₄H₁₄F₂O₃): 266.08, found 267.0 (M+H)⁺.

### Example 18

### In Vitro Assay

Human monocytic THP-1 cells were primed by IFNα (100 U/mL) for 22 h and then stimulated with 0.025% SAC in the presence of different concentrations of a test compound. The test compounds were prepared in DMSO and the final DMSO concentration was adjusted to 0.25% in all cell cultures, including the compound-free control. Supernatants from the cultures were removed after 24 h, and assayed for productions of IL-12 and TNFγ. Cell viability was assessed using the bioreduction of MTS [3-(4,5-dimethylthiazol-2-yl)-5-(3-carboxymethoxyphenyl)-2-(4-sulfophenyl)-2H-tetrazolium] (Promega, Madison, Wis.), with cell survival estimated as the ratio of the absorbance in compound-treated groups versus compound-free control. Human IL-12 p70 (heterodimer) and TNFα were assayed using ELISA kit from Endogen (Cambridge, Mass.), according to the manufacturer's instructions. The assay results are shown in Table 1.

TABLE 1

| | IC₅₀ (μM) | | CC₅₀ (μM) |
|---|---|---|---|
| | IL-12 | TNFα | Cell Toxicity |
| 1 | 30 | 40 | >100 |
| 2 | 30 | 30 | >100 |
| 3 | 70 | 40 | |
| 4 | 40 | 20 | >100 |
| 5 | 80 | 100 | >100 |
| 6 | 70 | 100 | >100 |
| 7 | 38 | 62 | >100 |
| 8 | 65 | 70 | >100 |
| 9 | 86 | 100 | >100 |
| 10 | 10 | 13 | 100 |
| 11 | 10 | 5 | 100 |
| 12 | 10 | 25 | 100 |
| 13 | 80 | 100 | >100 |
| 14 | 70 | 70 | >100 |
| 15 | 90 | 60 | >100 |
| 16 | 80 | 80 | >100 |
| 17 | 80 | 80 | >100 |
| 18 | 80 | 70 | >100 |
| 19 | 70 | 70 | >100 |
| 20 | 80 | 100 | >100 |
| 21 | 100 | 80 | >100 |

### OTHER EMBODIMENTS

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A compound of formula (I): 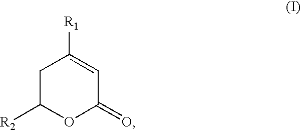
wherein
R₁ is H, OH, or C₂˜C₅ alkoxyl; and
R₂ is 2-phenyl ethyl, 2-phenyl ethenyl, 2-heteroaryl ethyl, or 2-heteroaryl ethenyl; in which the phenyl or the heteroaryl is optionally mono-, di-, or tri-substituted with R₃, R₄, or R₅; each of R₃, R₄, and R₅, independently, is Cl, F, Br, I, CN, C₁˜C₅ alkyl, C₁˜C₅ alkoxyl, C₃˜C₅ alkenyloxy, C₄˜C₆ cycloalkoxyl, C₄˜C₈ cycloalkyl alkoxyl, C₃˜C₅ alkoxy alkoxyl, or C₁˜C₄ alkoxy carbonyl.

2. The compound of claim 1, wherein R₁ is H.

3. The compound of claim 2, wherein R₂ is 2-phenyl ethyl.

4. The compound of claim 3, wherein each of R₃, R₄, and R₅, independently, is F or OCH₃.

5. The compound of claim 1, wherein R₁ is OH.

6. The compound of claim 5, wherein R₂ is 2-phenyl ethyl or 2-heteroaryl ethenyl.

7. The compound of claim 6, wherein each of R₃, R₄, and R₅, independently, is F.

8. The compound of claim 1, wherein R₁ is C₂˜C₅ alkoxyl.

9. The compound of claim 8, wherein R₂ is 2-phenyl ethyl.

10. A compound of formula (I): 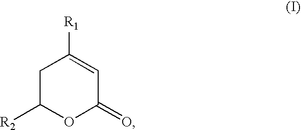
wherein
R₁ is OCH₃; and
R₂ is 2-phenyl ethyl or 2-phenyl ethenyl; in which the phenyl is mono-, di-, or tri-substituted with R₃, R₄, or R₅; each of R₃, R₄, and R₅, independently, is F, CN, or C₁˜C₄ alkoxy carbonyl.

11. The compound of claim 10, wherein each of R₃, R₄, and R₅, independently, is F.

12. The compound of claim 10, wherein each of R₃, R₄, and R₅, independently, is CN.

13. A compound of formula (I): 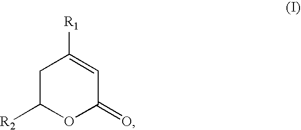
wherein
R₁ is OCH₃; and
R₂ is 2-heteroaryl ethyl or 2-heteroaryl ethenyl; in which the heteroaryl is optionally mono-, di-, or tri-substituted with R₃, R₄, or R₅; each of R₃, R₄, and R₅, independently, is Cl, F, Br, I, CN, C₁˜C₅ alkyl, C₁˜C₅ alkoxyl, C₃˜C₅ alkenyloxy, C₄˜C₆ cycloalkoxyl, C₄˜C₈ cycloalkyl alkoxyl, C₃˜C₅ alkoxy alkoxyl, or C₁˜C₄ alkoxy carbonyl.

14. The compound of claim 13, wherein R₂ is 2-heteroaryl ethyl.

15. A compound of formula (I): 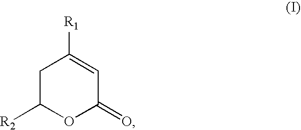
wherein
R₁ is H, OH, or C₂˜C₅ alkoxyl; and
R₂ is 2-phenyl ethyl, 2-phenyl ethenyl, 2-heteroaryl ethyl, or 2-heteroaryl ethenyl; in which the phenyl or the heteroaryl is optionally mono-, di-, or tri-substituted with R₃, R₄, or R₅; each of R₃, R₄, and R₅, independently, is Cl, F, Br, I, CN, C₁˜C₅ alkyl, C₁˜C₅ alkoxyl, C₃˜C₅ alkenyloxy, C₄˜C₆ cycloalkoxyl, C₄˜C₈ cycloalkyl alkoxyl, C₃˜C₅ alkoxy alkoxyl, or C₁˜C₄ alkoxy carbonyl; or
R₁ is OCH₃; and
R₂ is 2-phenyl ethyl or 2-phenyl ethenyl;
in which the phenyl is mono-, di-, or tri-substituted with R₃, R₄, or R₅; each of R₃, R₄, and R₅, independently, is F, CN, or C₁˜C₄ alkoxy carbonyl; or
R₁ is OCH₃; and
R₂ is 2-heteroaryl ethyl or 2-heteroaryl ethenyl;
in which the heteroaryl is optionally mono-, di-, or tri-substituted with R₃, R₄, or R₅; each of R₃, R₄, and R₅, independently, is Cl, F, Br, I, CN, C₁˜C₅ alkyl, C₁˜C₅ alkoxyl, C₃˜C₅ alkenyloxy, C₄˜C₆ cycloalkoxyl, C₄˜C₈ cycloalkyl alkoxyl, C₃˜C₅ alkoxy alkoxyl, or C₁˜C₄ alkoxy carbonyl.

16. The compound of claim 15, wherein the compound is any of:

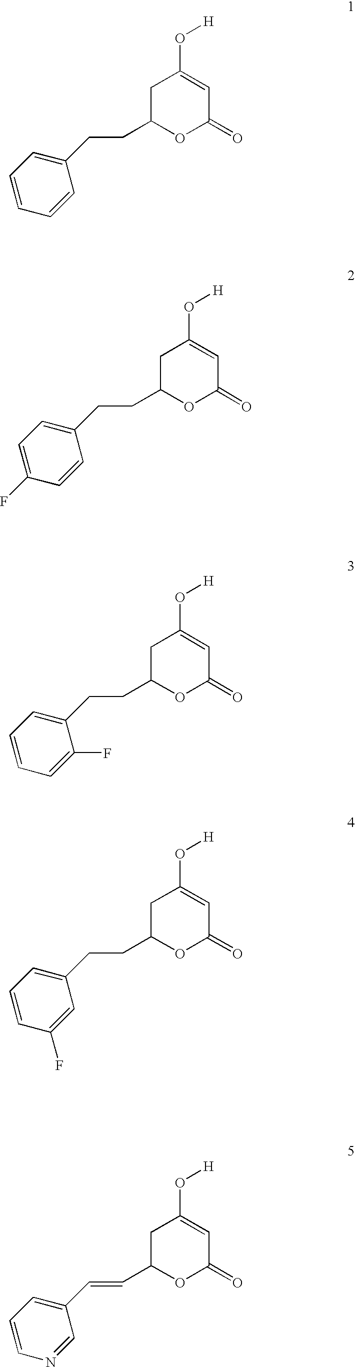

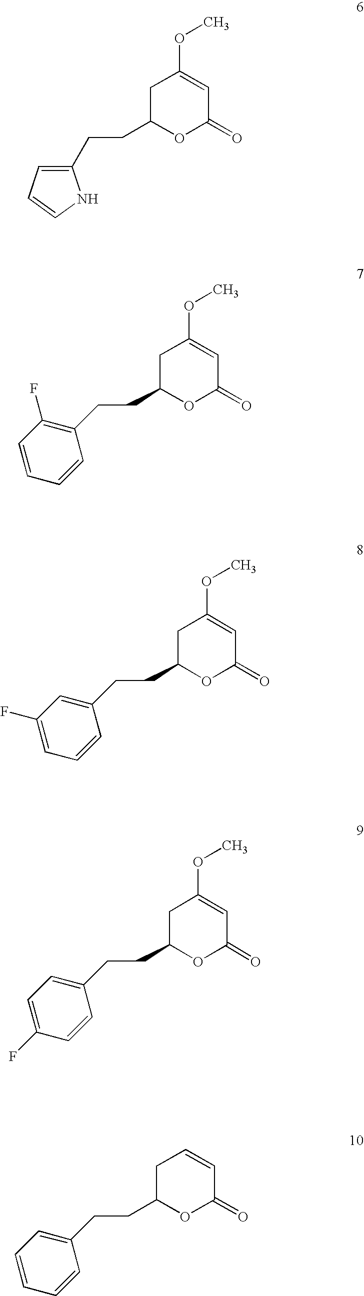

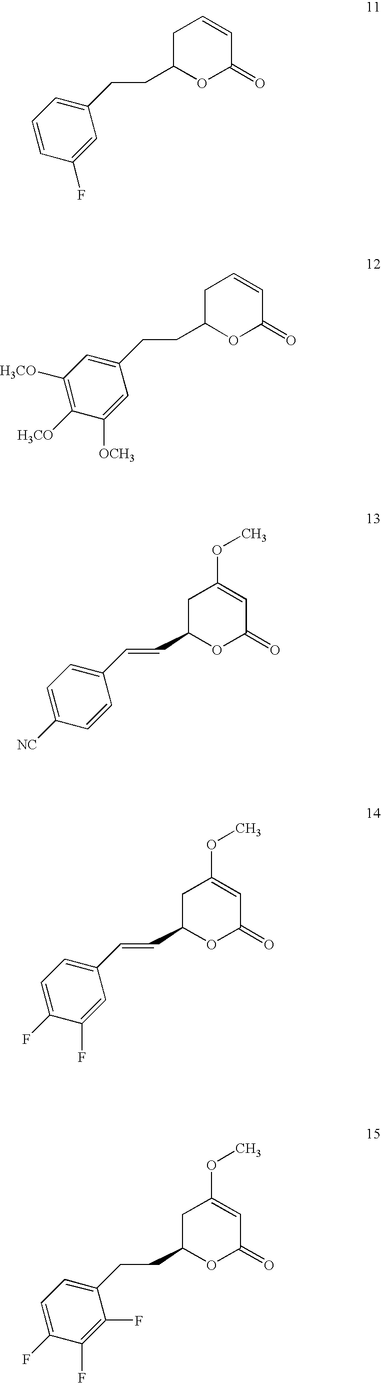

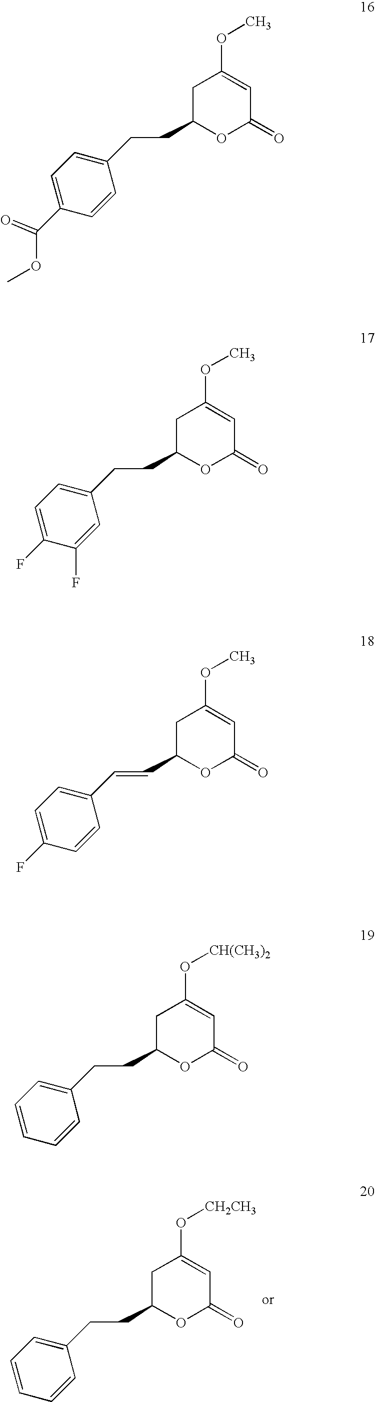

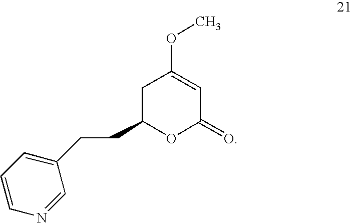

17. A pharmaceutical composition comprising a compound of claim 15 and a pharmaceutically acceptable carrier.

18. A method of making a compound of claim 15, comprising reacting a 5-R₂-5-hydroxy-3-oxo-pentanoic acid ester with a reagent to produce a compound of claim 15, wherein R₂ is 2-phenyl ethyl, 2-phenyl ethenyl, 2-heteroaryl ethyl, or 2-heteroaryl ethenyl.

19. A method of making a composition, comprising combining a compound of claim 15 and a pharmaceutically acceptable carrier.

20. A method of treating a neurodegenerative disorder, comprising administering to a subject in need thereof an effective amount of a compound of claim 15.

21. A method of eliciting an anticonvulsive or antiepileptic effect, comprising administering to a subject in need thereof an effective amount of a compound of claim 15.

22. A method of treating a neurological or psychiatric disorder, comprising administering to a subject in need thereof an effective amount of a compound of claim 15.

23. A method of eliciting an analgesic effect, comprising administering to a subject in need thereof an effective amount of a compound of claim 15.

24. A method of treating pain, comprising administering to a subject in need thereof an effective amount of a compound of claim 15.

25. A method of inhibiting IL-12 or TNFα production, comprising administering to a subject in need thereof an effective amount of a compound of claim 15.

26. A method of treating an IL-12 or TNFα-related disorder, comprising administering to a subject in need thereof an effective amount of a compound of claim 15.

27. A method of stimulating hair growth or retarding hair loss, comprising administering to a subject in need thereof an effective amount of a compound of claim 15.
